(19) United States
(12) Patent Application Publication
(10) Pub. No.: US 2023/0313115 A1
(43) Pub. Date: Oct. 5, 2023

(54) Cell Culturing System

(71) Applicants: Terumo Kabushiki Kaisha, Tokyo (JP); Terumo BCT, Inc., Lakewood, CO (US)

(72) Inventors: Masatsugu IGARASHI, Kanagawa (JP); Hirotaka OHASHI, Tokyo (JP); Andrew GLOOR, Lakewood, CO (US)

(21) Appl. No.: 18/206,186

(22) Filed: Jun. 6, 2023

Related U.S. Application Data
(63) Continuation of application No. PCT/JP2022/012948, filed on Mar. 22, 2022.

(30) Foreign Application Priority Data
Mar. 26, 2021 (JP) 2021-053044

(51) Int. Cl. C12M 1/36; C12M 1/12

(52) U.S. Cl. CPC C12M 41/48; C12M 25/10

(57) ABSTRACT

A cell culturing system is equipped with a plurality of processing units that perform culturing of cells, a plurality of reactor installation devices in which the plurality of processing units are capable of being respectively installed, a plurality of connection circuits connected respectively to the plurality of processing units, a plurality of circuit control devices which the plurality of connection circuits are capable of being respectively attached to and detached from, and a sensor device. The sensor device is used in common with respect to the plurality of processing units in order to measure components of the culture medium guided into the plurality of processing units. Each of the plurality of processing units includes a plurality of bioreactors.

### CROSS-REFERENCE TO RELATED APPLICATIONS

The present application is a continuation application of the International Patent Application No. PCT/JP2022/012948 filed on Mar. 22, 2022, which designated the U.S. and claims the benefit of priority from Japanese Patent Application No. JP2021-053044 filed on Mar. 26, 2021. The entire disclosures of the above-identified applications are incorporated herein by reference.

### FIELD

The present disclosure relates to a cell culturing system.

### BACKGROUND

A cell culturing device may be equipped with a reactor installation unit and a circuit control unit. The reactor installation unit may include a bioreactor in which cells are cultured. The circuit control unit may enable a connection circuit connected to the bioreactor to be attached and detached. The circuit control unit may be configured to supply cells and a culture medium from the connection circuit to the bioreactor and may be configured to move a collection of cultured cells from the bioreactor to the connection circuit.

It may be desirable to increase the amount of the cell culture, and in such instances, it may be necessary to prepare a plurality of the cell culturing devices. That is, it may be necessary to provide the same number of circuit control units as the number of bioreactors, thereby increasing the cost of the system.

The present disclosure provides a cell culturing system that is capable of efficiently increasing the amount of a cell culture while suppressing an increase in cost.

### SUMMARY

In at least one example embodiment, the present disclosure provides a cell culturing system. The cell culturing system may include a plurality of processing units that are configured to perform culturing of cells, a plurality of reactor installation devices in which the plurality of processing units are respectively installable, a plurality of connection circuits configured to be connected respectively to the plurality of processing units, and a plurality of circuit control devices which the plurality of connection circuits are respectively attachable to and detachable from. Each of the circuit control devices may be configured to supply the cells and also a culture medium from the plurality of connection circuits to the plurality of processing units and also to move a collection of cultured cells from the plurality of processing units to the plurality of connection circuits. The cell culturing system may also include a sensor device configured to be used in common with respect to the plurality of processing units in order to measure components of the culture medium guided into the plurality of processing units. Each of the plurality of processing units of the cell culturing system may include a plurality of bioreactors.

Since it is sufficient to prepare a circuit control device for each of the processing units, the number of the circuit control devices may be smaller than the number of the bioreactors. Thus, the amount of the cell culture can be efficiently increased while suppressing an increase in cost of preparing and using the of the cell culturing system. Further, since the sensor device is used in common by the plurality of processing units, the number of the sensor devices can be reduced, in comparison with a case in which a sensor device is provided for each of the plurality of processing units. Accordingly, it is possible to further suppress an increase in the cost of preparing and using the cell culturing system.

### BRIEF DESCRIPTION OF DRAWINGS

FIG. 1 is a schematic illustration of a cell culturing system according to at least one example embodiment of the present disclosure;

FIG. 2 is a circuit configuration diagram of the cell culturing system illustrated in FIG. 1;

FIG. 3 is a further circuit configuration diagram of the processing unit as illustrated in FIG. 2 and a surrounding periphery thereof;

FIG. 4 is a cross-sectional view of the tank device illustrated in FIG. 1;

FIG. 5 is a cross-sectional view taken along line V-V of FIG. 4;

FIG. 6 is a perspective view with partial omission of the cell culturing system shown in FIG. 1;

FIG. 7 is a perspective of the circuit control device illustrated in FIG. 6; and

FIG. 8 is a flowchart of a cell culturing method that uses the cell culturing system illustrated in FIG. 1.

### DETAILED DESCRIPTION

An example embodiment of a cell culturing system according to the present disclosure will be presented and described in detail below with reference to the accompanying drawings.

The cell culturing system 10 according to at least one example embodiment of the present invention may be a system for culturing or expanding cells that have been separated from living tissue.

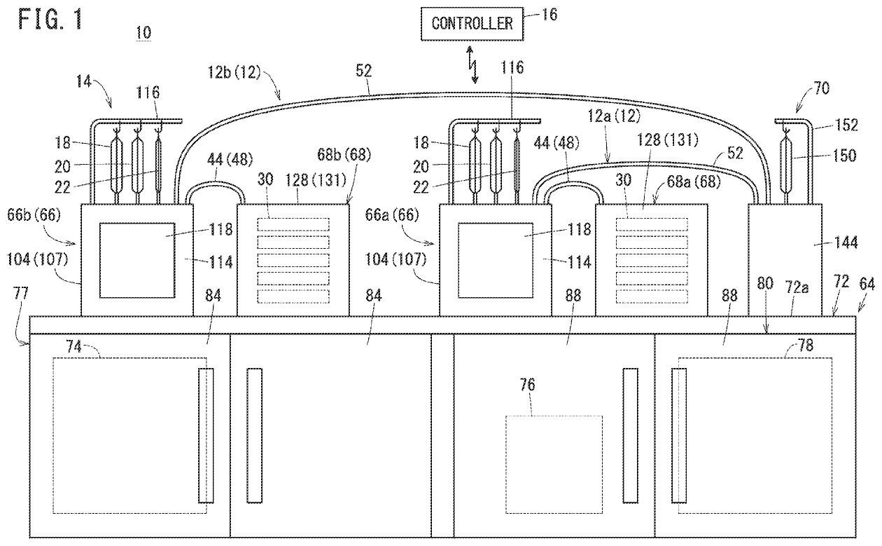

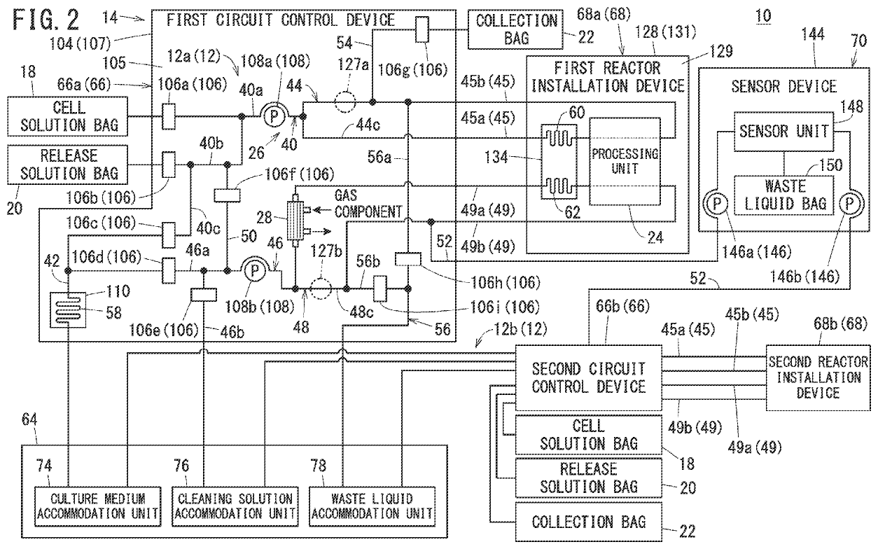

As illustrated in FIGS. 1 and 2, the cell culturing system 10 may include two cell culturing kits 12 in which liquids are capable of flowing, a cell culturing device 14 in which the two cell culturing kits 12 are set, and a controller 16.

The two cell culturing kits 12 may include a first cell culturing kit 12a and a second cell culturing kit 12b. The first cell culturing kit 12a and the second cell culturing kit 12b may be the same as each other.

The liquids that flow inside the cell culturing kits 12 may include a solution containing cells (hereinafter referred to as a cell solution), a culture medium (which may also be referred to as a culturing solution) in order to cause the cells to be expanded, a cleaning solution for cleaning the interior of the cell culturing kits 12, and/or a release solution for releasing the cells.

The cells may include cells from the blood (such as T cells and the like) and/or stem cells (such as ES cells, iPS cells, mesenchymal stem cells, and/or the like). An appropriate culture medium may be selected for the biological cells. For example, in at least one example embodiment, a culture medium may be prepared by adding various amino acids, vitamins, serum, and the like to a basic solution. The basic solution may include a buffered salt solution (BSS). The cleaning solution may include, for example, a buffer solution and/or a physiological saline solution. The buffer solution may include, for example, phosphate buffered salts (PBS), tris-buffered saline (TBS), and/or the like. The release solution may include, for example, trypsin and/or an EDTA solution. It should be appreciated, however, that the cell solution, the culture medium, the cleaning solution, and the release solution are not limited to the above examples.

As illustrated in FIG. 2, each of the cell culturing kits 12 may include a cell solution bag 18, a release solution bag 20, a collection bag 22, a processing unit 24, a connection circuit 26, and/or a gas exchanger 28.

The cell solution bag 18, the release solution bag 20, and/or the collection bag 22 may each include a flexible, soft resin material. The soft resin material may include, for example, polyvinyl chloride and/or polyolefin.

The cell solution bag 18 may be configured to carry or hold or receive the cell solution. The release solution bag 20 may be configured to carry or hold or receive the release solution. The collection bag 22 may be configured to carry or hold or receive the cultured cells. In a state prior to use, the collection bags 22 may be empty—that is, liquid is not yet accommodated in the interior thereof.

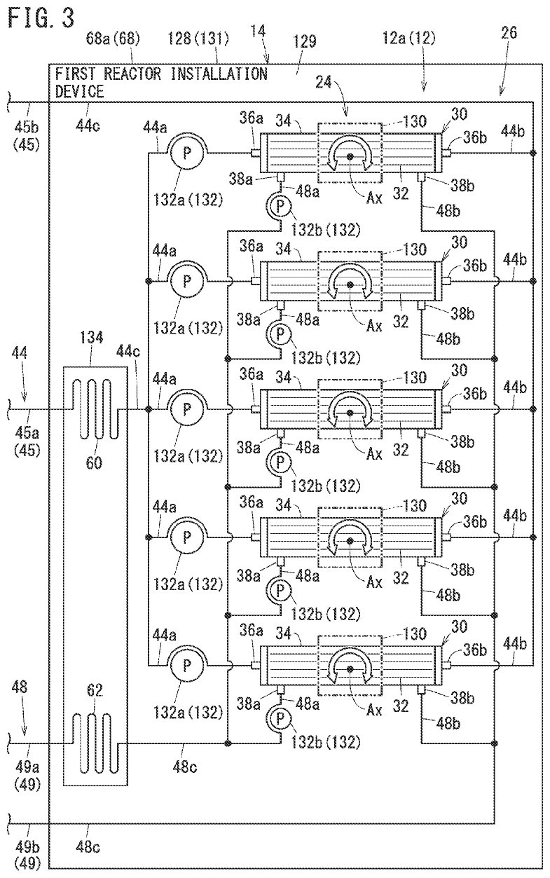

As illustrated in FIG. 3, the processing unit 24 may include five bioreactors 30 arranged in parallel. Each of the five bioreactors 30 may have the same configuration as each other. The five bioreactors 30 may differ, however, from each other in terms of the size and the shape thereof. Each of the bioreactors 30 may be configured as a hollow fiber type bioreactor. Each of the bioreactors 30 may be equipped with a large number of hollow fibers 32 and a cylindrical housing 34 in which the hollow fibers 32 are accommodated.

The hollow fibers 32 may extend in a longitudinal direction of the housing 34. Both ends of each of the hollow fibers 32 may be open. One end of each hollow fiber 32 may be fixed to one end of the housing 34. The other end of each hollow fiber 32 may be fixed to the other end of the housing 34. The wall that makes up each hollow fiber 32 may include a plurality of pores formed therewithin. The pores may enable communication between an intra capillary (IC) region or space and an extra capillary (EC) region or space. The IC region refers to the internal cavities of the hollow fibers 32. The EC region refers to an outer side of the hollow fibers 32 between the hollow fibers 32 and an interior surface of the housing 34. The diameter of the pores may be set to a size that allows small molecules (for example, water, ions, oxygen, lactate, etc.) to pass therethrough, while preventing the passage of larger molecules (for example, macromolecules like cells) therethrough. In at least one example embodiment, the diameter of the pores may be greater than or equal to about 0.005 micrometers to less than or equal to about 10 micrometers.

The hollow fibers 32 may include polyolefin resins and/or other polymeric materials. The polyolefin resins may include, for example, polypropylene, polyethylene, and the like. The polymer materials may include, for example, polysulfone, polyether sulfone, polyacrylonitrile, polytetrafluoroethylene, polystyrene, polymethylmethacrylate, cellulose acetate, cellulose triacetate, regenerated cellulose, and the like. It should be recognized, however, that the materials constituting the hollow fibers 32 are not limited to the above examples.

The housing 34 may include an IC inlet port 36a, an IC outlet port 36b, an EC inlet port 38a, and/or an EC outlet port 38b. The IC inlet port 36a may be provided on one end of the housing 34. The IC inlet port 36a may be configured to introduce liquids (e.g., a cell solution, a culture medium, a cleaning solution, and/or a release solution) into the IC region of the bioreactor 30. The liquids may be guided into the IC region of the bioreactor 32 using the connection circuit 26, and more specifically, an IC circulation circuit 44 of the connection circuit 26. The IC outlet port 36b may be provided on another end of the housing 34 away from the IC inlet port 36a. The IC outlet port 36b may be configured to allow the liquids that have flown through the IC region of the bioreactor 30 to be delivered to the connection circuit 26, and more specifically, the IC circulation circuit 44 of the connection circuit 26.

The EC inlet port 38a and the EC outlet port 38b may be provided on an outer circumferential surface of the housing 34. The EC inlet port 38a may be configured to introduce liquids (e.g., the culture medium and/or the cleaning solution) into an EC region of the bioreactor 30. The liquids may be guided into the EC region of the bioreactor 30 using the connection circuit 26, and more specifically, the EC circulation circuit 28 of the connection circuit 26. The EC outlet port 38b may be configured to allow the liquids that have flown through the EC region of the bioreactor 30 to be delivered to (the connection circuit 26, and more specifically, the EC circulation circuit 28 of the connection circuit 26.

As illustrated in FIG. 2, the connection circuit 26 may extended in the form of a line. The connection circuit 26 may be formed in a tubular shape using a soft resin material. In other embodiments, however, the connection circuit 26 may be formed, for example, by stacking two sheets in a thickness direction and joining (for example, by fusion bonding and/or sealing) a location thereof other than the portion that serves as the flow path. A wall portion (e.g., a non-sealed portion) forming the connection circuit 26 may be formed so as to project outwardly with respect to the sealed location in a manner that the connection circuit 26 can serve as a flow path that is opened in its natural state. Excess or surplus parts of the sheets on one or both sides of the flow path of the connection circuit 26 may be removed. In each instance, the connection circuit 26 may include an IC supply flow path 40, a culture medium supply line 42, an IC circulation circuit 44, an EC supply flow path 46, an EC circulation circuit 48, a connection line 50, a sampling line 52, a collection line 54, and/or a waste liquid flow path 56.

The IC supply flow path 40 may include a first IC supply line 40a, a second IC supply line 40b, and/or a third IC supply line 40c. One end of the first IC supply line 40a (e.g., a first end of the first IC supply line 40a) may be aseptically joined to the cell solution bag 18. The other end of the first IC supply line 40a (e.g., a second end of the first IC supply line 40a) may be connected to the IC circulation circuit 44. One end of the second IC supply line 40b (e.g., a first end of the second IC supply line 40b) may be aseptically joined to the release solution bag 20. The other end of the second IC supply line 40b (e.g., a second end of the second IC supply line 40b) may be connected to an intermediate location of the first IC supply line 40a. One end of the third IC supply line 40c (e.g., a first end of the third IC supply line 40c) may be connected to the culture medium supply line 42. The other end of the third IC supply line 40c (e.g., a second end of the third IC supply line 40c) may be connected to an intermediate location of the second IC supply line 40b.

When each of the cell culturing kits 12 may be set in the cell culturing device 14, one end of the culture medium supply line 42 (e.g., a first end of the culture medium supply line 42) may be aseptically joined with respect to a connection tube of a culture medium accommodation unit 74 of the cell culturing device 14. The other end of the culture medium supply line 42 (e.g., a second end of the culture medium supply line 42) may be connected to the third IC supply line 40c. In the culture medium supply line 42, a culture medium intermediate flow path 58 may be provided in order to raise the temperature of the culture medium (e.g., a cooled culture medium) that is delivered out from the culture medium accommodation unit 74 to a desired temperature. The culture medium intermediate flow path 58 may be disposed between the culture medium accommodation unit 74 and the processing unit 24.

As illustrated in FIGS. 2 and 3, the IC circulation circuit 44 may cause the liquid which is introduced from the IC supply flow path 40 into the IC circulation circuit 44 to be circulated in the IC region of each of the bioreactors 30. As illustrated in FIG. 3, the IC circulation circuit 44 may include five IC introduction lines 44a, five IC lead-out lines 44b, and/or an IC circulation line 44c.

The five IC introduction lines 44a may be connected to the IC inlet ports 36a of the five bioreactors 30. The five IC lead-out lines 44b may be connected to the IC outlet ports 36b of the five bioreactors 30. One end of the IC circulation line 44c (e.g., a first end of the IC circulation line 44c) may be connected to the five IC introduction lines 44a. The other end of the IC circulation line 44c (e.g., a second end of the IC circulation line 44c) may be connected to the five IC lead-out lines 44b. In the IC circulation line 44c, an IC intermediate flow path 60 may be provided in order to raise the temperature of the liquid flowing through the IC circulation line 44c to a desired temperature.

As illustrated in FIG. 2, the EC supply flow path 46 may include a first EC supply line 46a and a second EC supply line 46b. One end of the first EC supply line 46a (e.g., a first end of the first EC supply line 46a) may be connected to the culture medium supply line 42. The other end of the first EC supply line 46a (e.g., a second end of the first EC supply line 46a) may be connected to the EC circulation circuit 48. When the cell culturing kits 12 are each set in the cell culturing device 14, one end of the second EC supply line 46b (e.g., a first end of the second EC supply line 46b) may be aseptically joined with respect to a connection tube of a later-described cleaning solution accommodation unit 76 of the cell culturing device 14. The other end of the second EC supply line 46b (e.g., a second end of the second EC supply line 46b) may be connected to an intermediate location of the first EC supply line 46a.

As illustrated in FIGS. 2 and 3, the EC circulation circuit 48 may cause the liquid which is introduced from the EC supply flow path 46 into the EC circulation circuit 48 to be circulated in the EC region of each of the bioreactors 30. As illustrated in FIG. 3, the EC circulation circuit 48 may include five EC introduction lines 48a, five EC lead-out lines 48b, and/or an EC circulation line 48c.

The five EC introduction lines 48a may be connected to the EC inlet ports 38a of the five bioreactors 30. The five EC lead-out lines 48b may be connected to the EC outlet ports 38b of the five bioreactors 30. One end of the EC circulation line 48c (e.g., a first end of the EC circulation line 48c) may be connected to the five EC introduction lines 48a. The other end of the EC circulation line 48c (e.g., a second end of the EC circulation line 48c) may be connected to the five EC lead-out lines 48b. In the EC circulation line 48c, an EC intermediate flow path 62 may be provided in order to raise the temperature of the liquid flowing through the EC circulation line 48c to a desired temperature.

As illustrated in FIG. 2, the connection line 50 may be configured to connect the IC supply flow path 40 and the EC supply flow path 46 to each other. For example, one end of the connection line 50 (e.g., a first end of the connection line 50) may be connected to the second IC supply line 40b downstream of a connection of the second IC supply line 40b with the third IC supply line 40c. The other end of the connection line 50 (e.g., a second end of the connection line 50) may be connected to the first EC supply line 46a downstream of a connection of the first EC supply line 46a with the second EC supply line 46b.

The sampling line 52 may be a flow path configured to acquire a portion of the culture medium that has flowed through the EC region of each of the bioreactors 30. One end of the sampling line 52 (e.g., a first end of the sampling line 52) may be connected to the EC circulation line 48c downstream of the processing unit 24. When each of the cell culturing kits 12 is set in the cell culturing device 14, the other end of the sampling line 52 (e.g., a second end of the sampling line 52) may be aseptically joined with respect to a connection tube of a later-described sensor device 70 of the cell culturing device 14. In at least one example embodiment, in the set state, one end of the sampling line 52 may be provided in a circuit control device 66 (see FIG. 1). In at least one example embodiment, in the set state, the one end of the sampling line 52 may be provided in a reactor installation device 68.

The collection line 54 may be a flow path configured to guide the cultured cells from the IC circulation circuit 44 into the collection bag 22. One end of the collection line 54 (e.g., a first end of the collection line 54) may be connected to the IC circulation line 44c downstream of the processing unit 24. The other end of the collection line 54 (e.g., a second end of the collection line 54) may be aseptically joined with respect to the collection bag 22.

The waste liquid flow path 56 may be a flow path configured to guide a liquid usage of which has been completed (e.g., a waste liquid) to a later-described waste liquid accommodation unit 78 of the cell culturing device 14. The waste liquid flow path 56 may include an IC waste liquid line 56a and an EC waste liquid line 56b. One end of the IC waste liquid line 56a (e.g., a first end of the IC waste liquid line 56a) may be connected to the IC circulation line 44c between the processing unit 24 and a connected part with the collection line 54. When the cell culturing kits 12 are set in the cell culturing system 10, the other end of the IC waste liquid line 56a (e.g., a second end of the IC waste liquid line 56a) may be aseptically joined with respect to a connection tube of the waste liquid accommodation unit 78. One end of the EC waste liquid line 56b (e.g., a first end of the EC waste liquid line 56b) may be connected to the EC circulation line 48c between a connected part with the sampling line 52 and a connected part with the first EC supply line 46a. The other end of the EC waste liquid line 56b (e.g., a second end of the EC waste liquid line 56b) may be connected to the IC waste liquid line 56a.

The gas exchanger 28 may be disposed in the EC circulation line 48c between a connecting portion with the first EC supply line 46a and the EC intermediate flow path 62. The gas exchanger 28 may be configured to mix a predetermined gas component with the liquid (e.g., the culture medium) flowing through the EC circulation line 48c. As the gas component to be mixed, for example, there may be cited a gas component that approximates the mixing ratio of natural air (e.g., nitrogen N₂: 75%, oxygen O₂: 20%, and carbon dioxide CO₂: 5%).

The structure of the gas exchanger 28 is not particularly limited and, in the same manner as the bioreactor 30, a structure can be applied in which a plurality of hollow fibers 32 are provided inside a housing 34.

As illustrated in FIGS. 1 and 2, the cell culturing device 14 may include one tank device 64, two circuit control devices 66, two reactor installation devices 68, and/or one sensor device 70. The two circuit control devices 66 may include a first circuit control device 66a and a second circuit control device 66b. The two reactor installation devise 68 may include a first reactor installation device 68a and a second reactor installation device 68b.

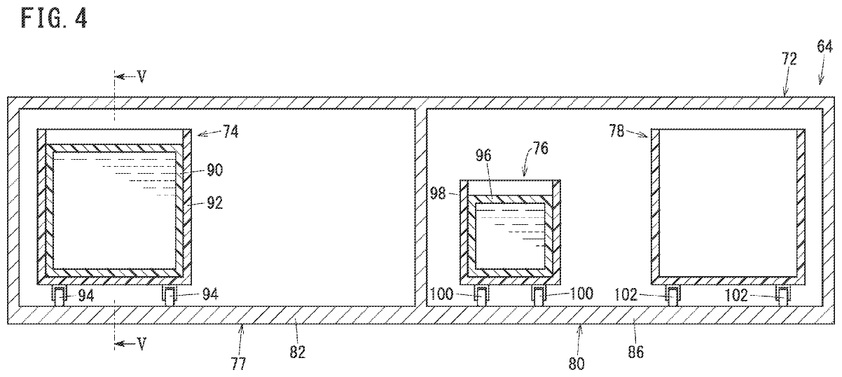

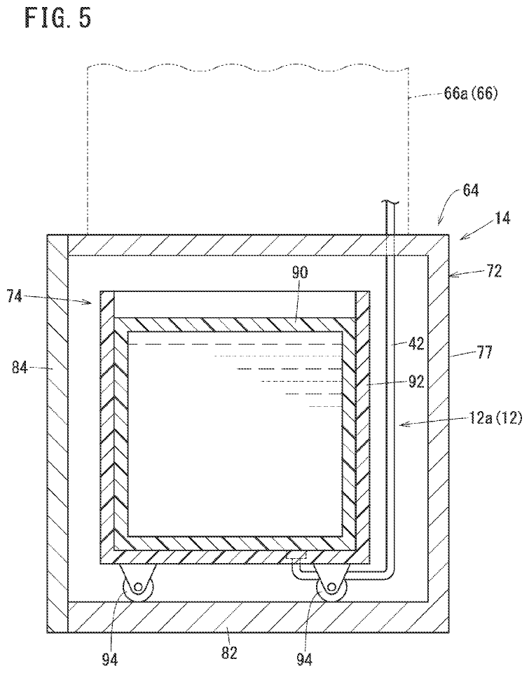

As illustrated in FIGS. 1 and 4, the tank device 64 may be equipped with a box-shaped pedestal 72 installed on a floor surface or the like, a culture medium accommodation unit 74 configured to accommodate the culture medium, a cleaning solution accommodation unit 76 configured to accommodate the cleaning solution, and a waste liquid accommodation unit 78 configured to accommodate the waste liquid. The pedestal 72 may include a first case portion 77 and a second case portion 80. The first case portion 77 may include a first case main body 82 in which the culture medium accommodation unit 74 can be arranged and a first door member 84 (see FIGS. 1 and 5) disposed on the front surface of the first case main body 82 so as to be capable of being opened and closed.

The first case portion 77 may be a cooling unit that is configured to cool the culture medium to a desired temperature (for example, greater than or equal to about 4 degrees Celsius to less than or equal to about 8 degrees Celsius). The second case portion 80 may include a second case main body 86 in which the cleaning solution accommodation unit 76 and the waste liquid accommodation unit 78 can be arranged and a second door member 88 (see FIG. 1) disposed on the front surface of the second case main body 86 so as to be capable of being opened and closed. The second case portion 80 does not have a cooling function.

As illustrated in FIGS. 4 and 5, the culture medium accommodation unit 74 may include a culture medium tank 90 that is formed in a box shape by a hard resin and a culture medium installation member 92 in which the culture medium tank 90 can be accommodated. The culture medium tank 90 may include a single-use product (e.g., a disposable product). However, in certain embodiments, the culture medium tank 90 may instead be a reusable product. The culture medium supply line 42 of each of the cell culturing kits 12 may be connected to the culture medium tank 90, in a state (referred to as a “set state”) where the cell culturing kits 12 are set in the cell culturing device 14. More specifically, the culture medium accommodation unit 74 (e.g., the culture medium tank 90) may be used in common with respect to two of the processing units 24 (e.g., two of the cell culturing kits 12) in order to supply the culture medium from the culture medium accommodation unit 74 to the two processing units 24 via two of the connection circuits 26.

The culture medium tank 90 may be capable of accommodating an amount of the culture medium necessary for culturing cells in the two processing units 24 (e.g., the two cell culturing kits 12). The culture medium tank 90 may be configured to accommodate an amount of the culture medium that is necessary for culturing cells by the two cell culturing kits 12 (which includes ten of the bioreactors 30) that are connected to the culture medium tank 90. For example, when 20 L of the culture medium is required for one of the bioreactors 30, the culture medium tank 90 may be configured to accommodate 200 L of the culture medium. Because a necessary amount of the culture medium from initiation to completion of cell culturing is accommodated in advance in the culture medium tank 90, there is no need to replace the culture medium accommodation unit 74 which improves efficiency. The culture medium may be accommodated in the culture medium tank 90 on a clean bench.

If the culture medium is stored at room temperature (for example, about 22 degrees Celsius) or in a bright location continuously over a period for which cell culturing is continued (for example, seven days or more), there may be a risk that the components of the culture medium (e.g., proteins, glutamines, and/or the like) may suffer from degeneration. However, in the current instance, because the culture medium is stored in the first case portion 77, which is a cool and dark place, degeneration of the components of the culture medium is effectively suppressed.

The culture medium installation member 92 include a hard resin. The culture medium installation member 92 may be a reusable product that is capable of being used again. The culture medium installation member 92 may be opened on an upper side. A plurality of rollers 94 (e.g., wheels) may be provided on a bottom surface of the culture medium installation member 92. In at least one example embodiment, the culture medium tank 90 may be arranged on an inner side of the culture medium installation member 92. As a result, the comparatively heavy culture medium accommodation unit 74 may be made to move smoothly due to the plurality of rollers 94. The culture medium accommodation unit 74 can be easily and efficiently taken out and inserted into the first case portion 77. The culture medium installation member 92 is not limited to the aforementioned configuration and may, in at least one example embodiment, include a trolley.

As illustrated in FIG. 4, the cleaning solution accommodation unit 76 may include a cleaning solution tank 96 that is formed in a box shape by a hard resin and a cleaning solution installation member 98 in which the cleaning solution tank 96 can be accommodated. The cleaning solution tank 96 may be a single-use product (e.g., a disposable product). However, in other embodiments, the cleaning solution tank 96 may be a reusable product. In a set state, the second EC supply line 46b of each of the cell culturing kits 12 may be connected to the cleaning solution tank 96. For example, the cleaning solution accommodation unit 76 (the cleaning solution tank 96) may be used in common with respect to two of the processing units 24 (two of the cell culturing kits 12) in order to supply the cleaning solution from the cleaning solution accommodation unit 76 to the two processing units 24 via the two connection circuits 26.

The cleaning solution tank 96 may be capable of accommodating an amount of the culture medium necessary for cleaning the two processing units 24 (the two cell culturing kits 12). The cleaning solution tank 96 may be configured to include an amount of the cleaning solution necessary for cleaning the two cell culturing kits 12 that are connected to the cleaning solution tank 96. In such instances, there is no need to replace the cleaning solution tank 96 during cell culturing which improves efficiency.

The cleaning solution installation member 98 may include a hard resin. The cleaning solution installation member 98 may be a reusable product that is capable of being used again. The cleaning solution installation member 98 may be opened on an upper side. A plurality of rollers 100 (e.g., wheels) may be provided on a bottom surface of the cleaning solution installation member 98. In at least one example embodiment, the cleaning solution tank 96 may be arranged on an inner side of the cleaning solution installation member 98. As a result, a relatively heavy cleaning solution accommodation unit 76 can be made to move smoothly due to the plurality of rollers 100. The cleaning solution accommodation unit 76 may be easily and efficiently taken out and inserted into the second case portion 80. The cleaning solution installation member 98 is not limited to the aforementioned configuration and may, in at least one example embodiment, include a trolley.

The waste liquid accommodation unit 78 may be formed in a box shape by a hard resin. The waste liquid accommodation unit 78 may be a reusable product that is capable of being used again. In certain embodiments, however, the waste liquid accommodation unit 78 may be a single-use product (e.g., a disposable product). In the set state, the waste liquid flow path 56 (the IC waste liquid line 56a) of each of the cell culturing kits 12 may be connected to the waste liquid accommodation unit 78. For example, the waste liquid accommodation unit 78 may be used in common with respect to the two processing units 24 (the two cell culturing kits 12) in order to discharge the waste liquid from the two processing units 24 into the waste liquid accommodation unit 78 via the two connection circuits 26.

The waste liquid accommodation unit 78 may be configured to accommodate the waste liquid that may be discharged from the two processing units 24 (the two cell culturing kits 12). For example, the waste liquid accommodation unit 78 may be sized to accommodate the waste liquid (solution) that is used by the two cell culturing kits 12 connected to the waste liquid accommodation unit 78. In such instances, there is no need to replace the waste liquid accommodation unit 78 during cell culturing, which improves efficiency.

A plurality of rollers 102 (wheels) may be provided on a bottom surface of the waste liquid accommodation unit 78 such that the waste liquid accommodation unit 78 can be moved smoothly. Thus, the waste liquid accommodation unit 78 can be easily and efficiently taken out and inserted into the second case portion 80.

The culture medium tank 90 and the cleaning solution tank 96 are not limited to the examples which include the hard resin. For example, in at least one example embodiment, the culture medium tank 90 and/or the cleaning solution tank 96 may include large capacity bags formed, for example, in a bag shape by a soft resin.

As illustrated in FIG. 1, the first circuit control device 66a, the first reactor installation device 68a, the second circuit control device 66b, the second reactor installation device 68b, and/or the sensor device 70 may be arranged on an upper surface 72a of the pedestal 72. The first circuit control device 66a and the first reactor installation device 68a, for example, may be disposed adjacent to each other. The second circuit control device 66b and the second reactor installation device 68b, for example, may be disposed adjacent to each other.

The connection circuit 26 of the first cell culturing kit 12a may be attached to and detached from the first circuit control device 66a. The first circuit control device 66a may be configured to supply the cells and the culture medium from the connection circuit 26 to the processing unit 24 and also to move a collection of the cultured cells from the processing unit 24 to the connection circuit 26.

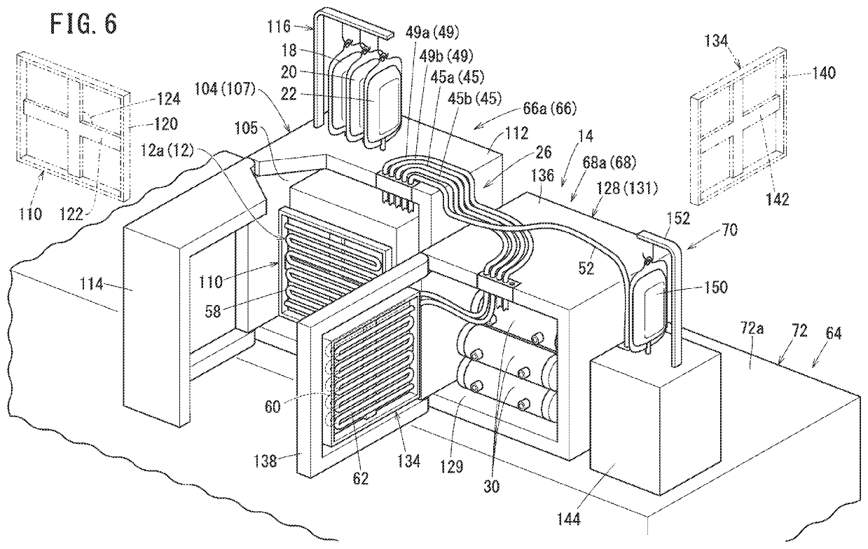

As illustrated in FIGS. 2 and 6, the first circuit control device 66a may include a box-shaped casing 104, a plurality of clamps 106, a plurality of pumps 108, and/or a first retaining member 110. As illustrated in FIG. 6, the casing 104 may include an internal space 105 in which the connection circuit 26 can be installed. The casing 104 may include a casing main body 112 and a casing door member 114 provided, for example, on a front surface of the casing main body 112 so as to be capable of being opened and closed.

The casing 104 may include a temperature control function configured to maintain the internal space 105 of the casing 104 at a desired temperature (for example, about 37 degrees Celsius). More specifically, the casing 104 may function as a temperature raising mechanism 107 configured to raise the temperature of the culture medium intermediate flow path 58. As illustrated in FIG. 1, a bag supporting member 116 configured to suspend a plurality of bags (e.g., the cell solution bag 18, the release solution bag 20, and/or the collection bag 22) may be provided on an upper surface of the casing 104. On an outer surface of the casing door member 114, a display unit 118 may provide for displaying a current processing step or the like of the cell culturing process (see FIG. 1).

As illustrated in FIG. 2, the plurality of clamps 106 may be ON/OFF valves that are configured to open and close internal flow paths of the lines (tubes) of the connection circuit 26 by pressing on wall portions of the lines (tubes) from an outer side. The first circuit control device 66a may include, as the plurality of clamps 106, a first clamp 106a, a second clamp 106b, a third clamp 106c, a fourth clamp 106d, a fifth clamp 106e, a sixth clamp 106f, a seventh clamp 106g, an eighth clamp 106h, and/or a ninth clamp 106i.

The first clamp 106a may be arranged so as to face the first IC supply line 40a in the set state and may be configured to open and close the internal flow path of the first IC supply line 40a. The second clamp 106b may be arranged so as to face the second IC supply line 40b in the set state and may be configured to open and close the internal flow path of the second IC supply line 40b. The third clamp 106c may be arranged so as to face the third IC supply line 40c in the set state and may be configured to open and close the internal flow path of the third IC supply line 40c.

The fourth clamp 106d may be arranged so as to face the first EC supply line 46a in the set state and may be configured to open and close the internal flow path of the first EC supply line 46a. The fifth clamp 106e may be arranged so as to face the second EC supply line 46b in the set state and may be configured to open and close the internal flow path of the second EC supply line 46b. The sixth clamp 106f may be arranged so as to face the connection line 50 in the set state and may be configured to open and close the internal flow path of the connection line 50.

The seventh clamp 106g may be arranged so as to face the collection line 54 in the set state and may be configured to open and close the internal flow path of the collection line 54. The eighth clamp 106h may be arranged so as to face the IC waste liquid line 56a in the set state and may be configured to open and close the internal flow path of the IC waste liquid line 56a. The ninth clamp 106i may be arranged so as to face the EC waste liquid line 56b in the set state and may be configured to open and close the internal flow path of the EC waste liquid line 56b.

The plurality of pumps 108 may be configured to apply a flowing force to the interior liquids by being rotated in a squeezing manner against the wall portions that form the lines (tubes) of the connection circuit 26. Each of the circuit control devices 66 may include, as the plurality of pumps 108, an IC supply pump 108a and/or an EC supply pump 108b.

In the set state, the IC supply pump 108a may be arranged so as to be in contact with a more downstream side than a portion on the first IC supply line 40a that is connected with the second IC supply line 40b and may be configured to impart a flowing force to the liquid flowing through the first IC supply line 40a in a direction toward the IC circulation circuit 44.

In the set state, the EC supply pump 108b may be arranged so as to be in contact with a more downstream side than the second EC supply line 46b in the first EC supply line 46a and may be configured to impart a flowing force to the liquid flowing through the second EC supply line 46b in a direction toward the EC circulation circuit 48.

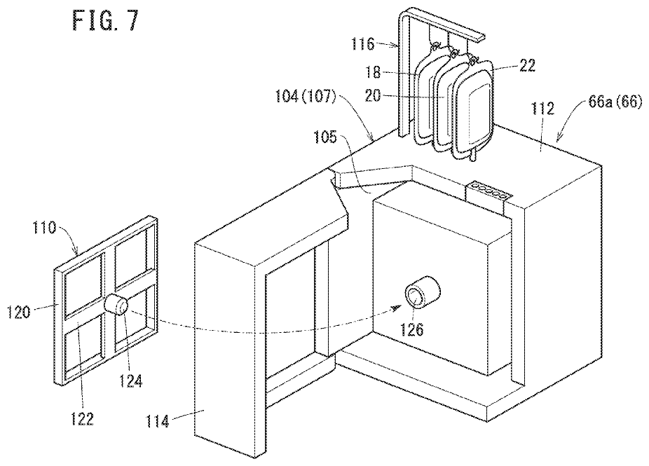

As illustrated in FIGS. 2 and 6, the first retaining member 110 may be configured to maintain the culture medium intermediate flow path 58 of the culture medium supply line 42 in a predetermined (meandering) shape. The first retaining member 110 may be provided in the internal space 105 of the casing 104. For example, as illustrated in FIGS. 6 and 7, the first retaining member 110 may include a rectangular first frame-shaped frame 120, a first inner side frame 122 disposed on the first frame-shaped frame 120, and/or an attachment member 124.

The first inner side frame 122 may be formed in the shape of a cross. The first inner side frame 122 may be connected to central portions of the respective sides of the first frame-shaped frame 120. As illustrated in FIG. 6, the culture medium intermediate flow path 58 may have a meandering shape and may be is locked in engagement by a non-illustrated locking member with respect to the first frame-shaped frame 120 and the first inner side frame 122. As illustrated in FIG. 7, the attachment member 124 may be a cylindrical columnar portion that projects out from a central portion of the first inner side frame 122. The attachment member 124 may be attached to a mounting member 126 provided in the casing 104. The number, size, shape, and position of the attachment member 124 are capable of being changed as appropriate.

As illustrated in FIG. 2, the length of the culture medium intermediate flow path 58 retained in the first retaining member 110 may be set to a length that is capable of allowing the culture medium to flow therethrough over a first temperature raising time period. In such instances, the first temperature raising time period may refer to a time period during which the temperature (for example, about 5 degrees Celsius) of the culture medium, which may be cooled in the culture medium accommodation unit 74, may be raised to a desired temperature (for example, about 37 degrees Celsius). Although not illustrated, it should be appreciated, that in at least one example embodiment, the first circuit control device 66a may be equipped with a pressure sensor, a liquid level sensor, and/or the like, none of which are shown.

In at least one example embodiment, the mounting member 126 (see FIG. 7) may be formed so as to be capable of rotatably supporting the bioreactors 30, and the first circuit control device 66a may further include an IC circulation pump 127a and/or an EC circulation pump 127b (see FIG. 2).

For example, in the case that a cell culture is desired to be implemented using a single bioreactor (e.g., in the case it is desired to perform a small amount of cell culturing), a cell culturing kit having only one bioreactor may be set in the first circuit control device 66a, and cell culturing can then be carried out. At this time, the aforementioned bioreactor may be set in the mounting member 126.

Further, the IC circulation pump 127a may be configured to impart a flowing force toward the bioreactor, to the liquid flowing through the IC circulation line of the aforementioned cell culturing kit. Furthermore, the EC circulation pump 127b may be configured to impart a flowing force toward the bioreactor, to the liquid flowing through the EC circulation line of the aforementioned cell culturing kit. Moreover, with cell culturing in which the cell culturing kits 12 having the plurality of (e.g., five) bioreactors 30 are used, the IC circulation pump 127a and/or the EC circulation pump 127b may not be used.

As illustrated in FIG. 2, the connection circuit 26 of the second cell culturing kit 12b may be set in the second circuit control device 66b. The configuration of the second circuit control device 66b may be the same as the configuration of the first circuit control device 66a.

As illustrated in FIGS. 3 and 6, the processing unit 24 of the first cell culturing kit 12a may be set in the first reactor installation device 68a. The first reactor installation device 68a may include a box-shaped reactor case portion 128, five reactor supporting members 130, a plurality of pumps 132, and/or a second retaining member 134. As illustrated in FIG. 6, the reactor case portion 128 may include an internal space 129 in which the processing unit 24 (the five bioreactors 30) are capable of being installed. The reactor case portion 128 may include a reactor case main body 136 and a door member 138 provided on a front surface of the reactor case main body 136 so as to be capable of being opened and closed. The reactor case portion 128 may include a temperature control function for maintaining the internal space 129 of the reactor case portion 128 at a desired temperature (for example, about 37 degrees Celsius). For example, the reactor case portion 128 may function as a temperature raising mechanism 131 for raising the temperature of the IC intermediate flow path 60.

As illustrated in FIG. 3, the reactor supporting members 130 may be disposed in the internal space 129 of the reactor case portion 128. The reactor supporting members 130 may be formed in a manner so that the bioreactors 30 can be attached and detached thereto. The reactor supporting members 130 support the bioreactors 30 to be capable of rotating about axes of rotation Ax. The axes of rotation Ax may be positioned at the center in the direction of extension of the bioreactors 30. The axes of rotation Ax may extend in a direction perpendicular to the direction of extension of the bioreactors 30.

The first reactor installation device 68a may include, as the plurality of pumps 132, five IC circulation pumps 132a and/or five EC circulation pumps 132b. The IC circulation pumps 132a may be arranged so as to be placed in contact with the IC introduction lines 44a in the set state and configured to impart a flowing force to the liquid flowing through the IC introduction lines 44a in a direction toward the bioreactors 30. The EC circulation pumps 132b may be arranged so as to be placed in contact with the EC introduction lines 48a in the set state and configured to impart a flowing force to the liquid flowing through the EC introduction lines 48a in a direction toward the bioreactors 30.

As illustrated in FIGS. 3 and 6, the second retaining member 134 may be configured to maintain the IC intermediate flow path 60 of the IC introduction lines 44a and the EC intermediate flow path 62 of the EC circulation line 48c, respectively, in a predetermined (meandering) shape. The second retaining member 134 may be provided in the internal space 129 of the reactor case portion 128. For example, as illustrated in FIG. 6, the second retaining member 134 may include a rectangular second frame-shaped frame 140 and a second inner side frame 142 disposed on an inner side of the second frame-shaped frame 140.

The second inner side frame 142 may be formed in the shape of a cross. The second inner side frame 142 may be connected to central portions of the respective sides of the second frame-shaped frame 140. Each of the IC intermediate flow path 60 and the EC intermediate flow path 62 may have a meandering shape and may be locked in engagement by a non-illustrated locking member with respect to the second frame-shaped frame 140 and the second inner side frame 142. The second retaining member 134 may be fixed to an inner surface of the door member 138.

As illustrated in FIGS. 1, 2, and 6, the first reactor installation device 68a may be disposed separately from the first circuit control device 66a. Therefore, in the set state, as illustrated in FIGS. 2 and 6, the first cell culturing kit 12a may include IC outer side flow paths 45 and EC outer side flow paths 49, which are positioned on an outer side of the first circuit control device 66a and the first reactor installation device 68a. The first cell culturing kit 12a may include, as the IC outer side flow paths 45, a first IC outer side flow path 45a and a second IC outer side flow path 45b. As illustrated in FIG. 2, the first IC outer side flow path 45a may be positioned in a section in the IC circulation line 44c between a connected part with the first IC supply line 40a and the IC intermediate flow path 60. The second IC outer side flow path 45b may be positioned in a section in the IC circulation line 44c between the processing unit 24 and a connected part with the IC waste liquid line 56a.

The liquid flowing through the IC circulation line 44c may be cooled at the positions of the first IC outer side flow path 45a and the second IC outer side flow path 45b. For example, at the positions of the first IC outer side flow path 45a and the second IC outer side flow path 45b, the liquid flowing through the IC circulation line 44c may be subjected to cooling to room temperature (for example, about 30 degrees Celsius).

The length of the IC intermediate flow path 60 retained in the second retaining member 134 may be set to a length that is capable of allowing the culture medium to flow therethrough over a second temperature raising time period. The second temperature raising time period may refers to a time period during which the temperature (for example, about 30 degrees Celsius) of the liquid, which is cooled in the first IC outer side flow path 45a or the second IC outer side flow path 45b when flowing through the IC circulation line 44c, may be raised to a desired temperature (the temperature of the internal space 129 of the reactor case portion 128).

Further, the first cell culturing kit 12a may include, as the EC outer side flow paths 49, a first EC outer side flow path 49a and a second EC outer side flow path 49b. The first EC outer side flow path 49a may be positioned in a section in the EC circulation line 48c between the gas exchanger 28 and the EC intermediate flow path 62. The second EC outer side flow path 49b may be positioned in a section, in the EC circulation line 48c, between the processing unit 24 and a connected part with the EC waste liquid line 56b.

The liquid flowing through the EC circulation line 48c may be cooled at the positions of the first EC outer side flow path 49a and the second EC outer side flow path 49b. For example, at the positions of the first EC outer side flow path 49a and the second EC outer side flow path 49b, the liquid (culture medium) flowing through the EC circulation line 48c may be subjected to cooling to room temperature (for example, about 30 degrees Celsius).

The length of the EC intermediate flow path 62 retained in the second retaining member 134 may be set to a length that is capable of allowing liquid to flow therethrough over a third temperature raising time period. The third temperature raising time period may refer to a time period during which the temperature (for example, about 30 degrees Celsius) of the liquid, which is cooled in the first EC outer side flow path 49a or the second EC outer side flow path 49b when flowing through the EC circulation line 48c, may be raised to a desired temperature (the temperature of the internal space 129 of the reactor case portion 128).

The processing unit 24 of the second cell culturing kit 12b may be set in the second reactor installation device 68b. The configuration of the second reactor installation device 68b may be the same as the configuration of the first reactor installation device 68a.

As illustrated in FIG. 2, in the set state, the sensor device 70 may be connected to the first cell culturing kit 12a and the second cell culturing kit 12b. The sensor device 70 may include a box-shaped sensor case portion 144 (see FIGS. 1 and 6), two pumps 146, a sensor unit 148, and/or a waste liquid bag 150. A bag supporting member 152 may be configured to suspend the waste liquid bag 150 and may be provided on an upper surface of the sensor case portion 144 (see FIGS. 1 and 6). The two pumps 146 and the sensor unit 148 may be disposed inside the sensor case portion 144.

The pumps 146 may be configured in the same manner as the pumps 108 described above. The sensor device 70 may include, as the two pumps 146, a first sampling pump 146a and a second sampling pump 146b. The first sampling pump 146a may be arranged so as to be placed in contact with the sampling line 52 of the first cell culturing kit 12a in the set state and may be configured to impart a flowing force to the liquid (the culture medium) flowing through the aforementioned sampling line 52 in a direction toward the sensor unit 148. The second sampling pump 146b may be arranged so as to be placed in contact with the sampling line 52 of the second cell culturing kit 12b in the set state and may be configured to impart a flowing force to the liquid (the culture medium) flowing through the aforementioned sampling line 52 in a direction toward the sensor unit 148.

The sensor unit 148 may be configured to measure the components (concentrations of PH, O₂, CO₂, glucose, lactic acid, and/or the like) of the culture medium that is guided by the sampling line 52. After measurement of the components by the sensor unit 148 is completed, the culture medium may be discharged into the waste liquid bag 150.

In the cell culturing device 14, the sensor device 70 (the sensor unit 148 and the waste liquid bag 150) may be used in common by the first cell culturing kit 12a and the second cell culturing kit 12b. Further, the tank device 64 may be used in common by the first cell culturing kit 12a and the second cell culturing kit 12b.

As illustrated in FIG. 1, the controller 16 may include a computer having a processor, a memory, and an input/output interface. By the processor executing a program that is stored in the memory, the controller 16 may perform a comprehensive control of the system as a whole. The controller 16 may be connected to the first circuit control device 66a, the first reactor installation device 68a, the second circuit control device 66b, the second reactor installation device 68b, and/or the sensor device 70, by way of a communication means including a wired communication, a wireless communication, a network, or a combination thereof.

For example, based on control signals from the controller 16, the first circuit control device 66a and/or the second circuit control device 66b may respectively control operations of the plurality of clamps 106 and the plurality of pumps 108. Based on control signals from the controller 16, the first reactor installation device 68a and the second reactor installation device 68b may respectively control operations of the plurality of IC circulation pumps 132a and the plurality of EC circulation pumps 132b, together with controlling rotational operation of each of the bioreactors 30.

Based on a control signal from the controller 16, the sensor unit 148 may be configured to acquire (samples) the culture medium flowing through the first cell culturing kit 12a or the second cell culturing kit 12b and may be configured to measure the components of the acquired culture medium.

Further, the sensor unit 148 may be configured to transmit measurement results to the controller 16. On the basis of the measurement results, the controller 16 may estimate the number of cells that were cultured in the first cell culturing kit 12a and the second cell culturing kit 12b. Based on measurement results from the sensor device 70, the controller 16 feedback may control operations of the first circuit control device 66a, the first reactor installation device 68a, the second circuit control device 66b, and/or the second reactor installation device 68b.

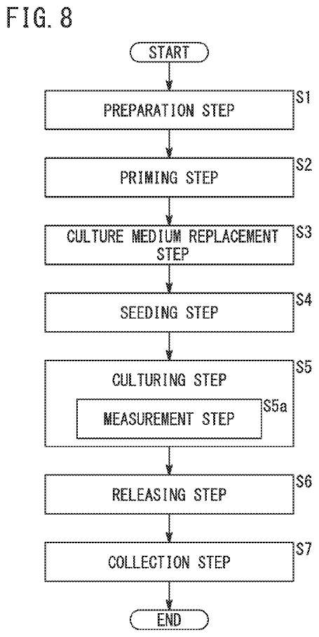

As illustrated in FIG. 8, the cell culturing method may include a preparation step, a priming step, a culture medium replacement step, a seeding step, a culturing step, a releasing step, and/or a collection step.

First, in the preparation step (step S1), as illustrated in FIGS. 2 and 8, the culture medium accommodation unit 74 may arranged on the first case portion 77 and the cleaning solution accommodation unit 76 and the waste liquid accommodation unit 78 being arranged in the second case portion 80. In addition, the processing unit 24 (the five bioreactors 30) of the first cell culturing kit 12a may be installed in the first reactor installation device 68a, and the connection circuit 26 of the first cell culturing kit 12a may be set in the first circuit control device 66a. At this time, a plurality of bags (e.g., the cell solution bag 18, the release solution bag 20, and/or the collection bag 22) of the first cell culturing kit 12a may be suspended from the bag supporting member 116 of the first circuit control device 66a. Further, the connection circuit 26 of the first cell culturing kit 12a may be aseptically joined to each of the culture medium accommodation unit 74, the cleaning solution accommodation unit 76, the waste liquid accommodation unit 78, and/or the sensor unit 148.

Subsequently, the processing unit 24 (the five bioreactors 30) of the second cell culturing kit 12b may be installed in the second reactor installation device 68b, and the connection circuit 26 of the second cell culturing kit 12b may be set in the second circuit control device 66b. At this time, a plurality of bags (e.g., the cell solution bag 18, the release solution bag 20, and/or the collection bag 22) of the second cell culturing kit 12b may be suspended from the bag supporting member 116 of the second circuit control device 66b. Further, the connection circuit 26 of the second cell culturing kit 12b may be aseptically joined to each of the culture medium accommodation unit 74, the cleaning solution accommodation unit 76, the waste liquid accommodation unit 78, and/or the sensor unit 148.

Thereafter, in the priming step (step S2), the circuit control devices 66 and the reactor installation devices 68 may be configured to drive predetermined ones of the clamps 106 and the pumps 108 and 132 thereby guiding the cleaning solution of the cleaning solution accommodation unit 76 to the connection circuit 26 and to each of the bioreactors 30. Consequently, the interior of the connection circuit 26 and the interior (the IC region and the EC region) of each of the bioreactors 30 may be filled with the cleaning solution. At this time, air existing inside the connection circuit 26 and the bioreactors 30 may be discharged into the waste liquid accommodation unit 78 together with the cleaning solution.

In addition, in the culture medium replacement step (step S3), the circuit control devices 66 and the reactor installation devices 68 may be configured to drive predetermined ones of the clamps 106 and the pumps 108 and 132 thereby guiding the culture medium of the culture medium accommodation unit 74 to the connection circuit 26 and to each of the bioreactors 30. Consequently, the cleaning solution existing in the interior of the connection circuit 26 and the interior (the IC region and the EC region) of each of the bioreactors 30 may be replaced by the culture medium.

Next, in the seeding step (step S4), the circuit control device 66 and the reactor installation device 68 may be configured to drive predetermined ones of the clamps 106 and the pumps 108 and 132 thereby supplying the cell solution of the cell solution bag 18 to the IC region of each of the bioreactors 30. For example, the cell solution that is guided from the cell solution bag 18 into the IC circulation line 44c via the first IC supply line 40a may be divided into five IC introduction lines 44a and guided into the IC region of each of the bioreactors 30 (see FIG. 3). At this time, since the five IC circulation pumps 132a impart a flowing force to the liquid (the cell solution) flowing through the five IC introduction lines 44a, the cell solution may be supplied to the five bioreactors 30 in a substantially uniform manner.

Thereafter, in the culturing step (step S5), the circuit control device 66 and the reactor installation device 68 may be configured to drive predetermined ones of the clamps 106 and the pumps 108 and 132, thereby supplying the culture medium in the culture medium accommodation unit 74 to the IC region and the EC region of each of the bioreactors 30, whereby the cells are cultured (expanded) inside the hollow fibers 32 of the bioreactors 30. Supplying of the culture medium to the IC region of each of the bioreactors 30 and supplying of the culture medium to the EC region of each of the bioreactors 30 may be carried out simultaneously, or may be carried out separately. Further, in the culturing step, the culture medium may be supplied only to the EC region of each of the bioreactors 30, without being supplied to the IC region of each of the bioreactors 30.

For example, in the culturing step, the culture medium, which is at a low temperature (for example, 5 degrees Celsius) inside the culture medium accommodation unit 74, may flow through the culture medium supply line 42 and may be guided from the tank device 64 into the culture medium intermediate flow paths 58 which are disposed in the internal spaces 105 of the casings 104 of the circuit control devices 66. The temperature of the culture medium flowing through the culture medium intermediate flow paths 58 may be raised to a desired temperature (for example, about 37 degrees Celsius).

In addition, in the case that the culture medium is supplied to the IC region of each of the bioreactors 30, the culture medium, which is raised in temperature in the culture medium intermediate flow path 58, may be introduced into the IC circulation line 44c via the third IC supply line 40c, the second IC supply line 40b, and/or the first IC supply line 40a. The temperature of the culture medium introduced into the IC circulation line 44c may be lowered (for example, is lowered to about 30 degrees Celsius) when flowing through the first IC outer side flow path 45a.

Thereafter, the culture medium the temperature of which has been lowered may be guided into the IC intermediate flow path 60 provided in the internal space 129 of the reactor case portion 128. The temperature of the culture medium flowing through the IC intermediate flow path 60 may be raised to a desired temperature (for example, about 37 degrees Celsius). The culture medium that has flowed through the IC intermediate flow path 60 may branch into the five IC introduction lines 44a and may be guided into the IC region of each of the bioreactors 30, whereby the culture medium in the IC region of each of the bioreactors 30 may be replaced by a new culture medium. Consequently, nutrients such as oxygen and the like may be efficiently supplied to the cells that are seeded on the inner surfaces of the hollow fibers 32 in each of the bioreactors 30.

Further, in the culturing step, the culture medium may circulate inside the IC circulation circuit 44. At this time, although the temperature of the culture medium may be lowered when flowing through the first IC outer side flow path 45a and the second IC outer side flow path 45b, since the temperature is raised in the IC intermediate flow path 60, the temperature of the culture medium supplied to the IC region of each of the bioreactors 30 may be maintained at the desired temperature.

Further, in the case that the culture medium is supplied to the EC region of each of the bioreactors 30, the culture medium, which is raised in temperature in the culture medium intermediate flow path 58, may be introduced into the EC circulation line 48c via the first EC supply line 46a. The culture medium that is introduced into the EC circulation line 48c, after having passed through the gas exchanger 28, may be lowered in temperature (for example, is lowered to about 30 degrees Celsius) when flowing through the first EC outer side flow path 49a.

Thereafter, the culture medium the temperature of which has been lowered may be guided into the EC intermediate flow path 62 provided in the internal space 129 of the reactor case portion 128. The temperature of the culture medium flowing through the EC intermediate flow path 62 may be raised to a desired temperature (for example, about 37 degrees Celsius). The culture medium that has flowed through the EC intermediate flow path 62 may branch into the five EC introduction lines 48a and may be guided to the EC region of each of the bioreactors 30. In the bioreactors 30, exchange of nutrients and the like may be carried out between the culture medium in the IC region and the culture medium in the EC region. Consequently, nutrients such as oxygen and the like may be efficiently supplied to the cells that are seeded on the inner surfaces of the hollow fibers 32 in each of the bioreactors 30.

Further, in the culturing step, the culture medium may circulate inside the EC circulation circuit 48. At this time, although the temperature of the culture medium is lowered when flowing through the first EC outer side flow path 49a and the second EC outer side flow path 49b, since the temperature is raised in the EC intermediate flow path 62, the temperature of the culture medium supplied to the EC region of each of the bioreactors 30 may be maintained at the desired temperature. Further, the culture medium circulating in the EC circulation circuit 48 may be subjected to gas exchange when flowing through the gas exchanger 28. Therefore, the culture medium in which desired gas components are included may be supplied to the EC region of each of the bioreactors 30.

Furthermore, the culturing step may include a measurement step (step S5a). In the measurement step, by driving the pumps 146, the sensor device 70 may be configured to guide the culture medium flowing through a portion on the downstream side of the processing unit 24 within the EC circulation line 48c, to the sensor unit 148. The sensor unit 148 measures the components of the culture medium (the culture medium inside the processing unit 24). The measurement results of the sensor unit 148 may be transmitted to the controller 16. Based on the measurement results, the controller 16 may be configured to determine points in time (a timing), an interval or time period, a number of times, or the like for the culture medium to be exchanged. After the measurements by the sensor unit 148 are completed, the culture medium may be discharged into the waste liquid bag 150. The points in time (the timing) and the number of times or the like that the measurement step is executed during the culturing step can be appropriately set.

Upon completion of the culturing step, in the releasing step (step S6), the circuit control device 66 and the reactor installation device 68 may be configured to drive predetermined ones of the clamps 106 and the pumps 108 and 132 thereby guiding the release solution to the IC region of each of the bioreactors 30. Consequently, the cells that were cultured (expanded) in the IC region of each of the bioreactors 30 may be released from the inner surfaces of the hollow fibers 32.

Subsequently, in the collection step (step S7), the circuit control devices 66 and the reactor installation devices 68 may be configured to drive predetermined ones of the clamps 106 and the pumps 108 and 132 thereby guiding the cells that were released off in the releasing step from each of the bioreactors 30 into the collection bag 22, while supplying the culture medium to the IC region of each of the bioreactors 30. Upon completion of the collection step, operations of the cell culturing method for the present time may be brought to an end.

The cell culturing system 10 may be equipped with the plurality of processing units 24 that are configured to perform culturing of cells; the plurality of reactor installation devices 68 in which the plurality of processing units 24 may be capable of being respectively installed; the plurality of connection circuits 26 that may be connected respectively to the plurality of processing units 24; the plurality of circuit control devices 66 which the plurality of connection circuits 26 may be capable of being respectively attached to and detached from, and which are capable of supplying the cells and the culture medium from the plurality of connection circuits 26 to the plurality of processing units 24 and also moving a collection of cultured cells from the plurality of processing units 24 to the plurality of connection circuits 26; and the sensor device 70 which is used in common with respect to the plurality of processing units 24, in order to measure the components of the culture medium that may be guided into the plurality of processing units 24. Each of the plurality of processing units 24 may include a plurality of bioreactors 30.

In accordance with such a configuration, since it is sufficient to prepare the circuit control device 66 for each of the processing units 24 (each unit including the plurality of bioreactors 30), the number of the circuit control devices 66 becomes smaller than the number of the bioreactors 30. Thus, the amount of the cell culture can be efficiently increased while suppressing an increase in cost. Further, since the sensor device 70 may be used in common by the plurality of processing units 24, the number of the sensor devices 70 can be reduced, in comparison with a case in which a sensor device 70 is provided for each of the plurality of processing units 24. Accordingly, it is possible to further suppress an increase in the cost of the cell culturing system 10.

The sensor device 70 may be connected to the plurality of connection circuits 26, in a set state in which the plurality of processing units 24 are installed respectively in the plurality of reactor installation devices 68.

The cell culturing device 14 may include the tank device 64 having the culture medium accommodation unit 74 in which the culture medium may be accommodated. The culture medium accommodation unit 74 may be used in common with respect to the plurality of processing units 24, in order to supply the culture medium from the culture medium accommodation unit 74 to the plurality of processing units 24 via the plurality of connection circuits 26.

In accordance with such a configuration, since the culture medium accommodation unit 74 may be used in common by the plurality of processing units 24 (the plurality of cell culturing kits 12), the number of the culture medium accommodation units 74 can be reduced, as compared with a case in which a culture medium accommodation unit 74 is provided for each of the plurality of processing units 24.

The culture medium accommodation unit 74 may be capable of accommodating an amount of the culture medium necessary for culturing cells in the plurality of processing units 24.

In accordance with such a configuration, even if a large amount of the culture medium is required for cell culturing using the plurality of processing units 24 (the plurality of bioreactors 30), it becomes unnecessary to replace the culture medium accommodation unit 74 during cell culturing. Therefore, cell culturing can be performed smoothly and efficiently.

The tank device 64 may include the waste liquid accommodation unit 78 in which the waste liquid may be accommodated. The waste liquid accommodation unit 78 may be used in common with respect to the plurality of processing units 24, in order to discharge the waste liquid from the plurality of processing units 24 into the waste liquid accommodation unit 78 via the plurality of connection circuits 26.

In accordance with such a configuration, since the waste liquid accommodation unit 78 may be used in common by the plurality of processing units 24 (the plurality of cell culturing kits 12), the number of the waste liquid accommodation units 78 can be reduced, as compared with a case in which a waste liquid accommodation unit 78 is provided for each of the plurality of processing units 24.

The tank device 64 may include the cleaning solution accommodation unit 76 in which the cleaning solution is accommodated. The cleaning solution accommodation unit 76 may be used in common with respect to the plurality of processing units 24, in order to supply the cleaning solution from the cleaning solution accommodation unit 76 to the plurality of processing units 24 via the plurality of connection circuits 26.

In accordance with such a configuration, since the cleaning solution accommodation unit 76 may be used in common by the plurality of processing units 24 (the plurality of cell culturing kits 12), the number of the cleaning solution accommodation units 76 can be reduced, as compared with a case in which a cleaning solution accommodation unit 76 is provided for each of the plurality of processing units 24.

The cleaning solution accommodation unit 76 may be capable of accommodating an amount of the cleaning solution necessary for cleaning the plurality of processing units 24.

In accordance with such a configuration, even if a large amount of the cleaning solution is required for cell culturing using the plurality of processing units 24 (the plurality of cell culturing kits 12), it becomes unnecessary to replace the cleaning solution accommodation unit 76 during cell culturing. Therefore, cell culturing can be performed smoothly and efficiently.

The sensor device 70 may include the sensor unit 148 that is configured to measure the components of the culture medium and the plurality of pumps 146 that guide the culture medium, which has flowed through the plurality of processing units 24, to the sensor unit 148.

In accordance with such a configuration, the culture medium that has passed through the plurality of processing units 24 can be efficiently guided to the sensor unit 148.

The cell culturing system 10 may further include the controller 16 that controls operation of the plurality of circuit control devices 66. The controller 16 feedback may control operation of the plurality of circuit control devices 66 based on a measurement result of the sensor device 70.

Each of the plurality of bioreactors 30 may include a plurality of the hollow fibers 32.

The present disclosure is not limited to the above-described embodiments, various modifications may be adopted within a range that does not depart from the essence and gist of the present invention.

The number of the bioreactors 30 that the reactor installation device 68 can accommodate therein is not limited to five, and may be two, three, four, or six or more. In the cell culturing system 10, the circuit control devices 66 and the reactor installation devices 68 may be provided, respectively, in a number of three or more. In this case, the tank devices 64 and the sensor devices 70 may be provided, respectively, in a number of two or more.

In the cell culturing system 10, the IC intermediate flow path 60 or the EC intermediate flow path 62 may be omitted. Further, in the cell culturing system 10, both the IC intermediate flow path 60 and the EC intermediate flow path 62 may be omitted, and together therewith, the second retaining member 134 may be omitted. Furthermore, in the cell culturing system 10, the culture medium intermediate flow path 58 and the first retaining member 110 may be omitted.

In at least one example embodiment, the cell culturing system (10) is disclosed, which may include the plurality of processing units (24) that perform culturing of cells; the plurality of reactor installation devices (68) in which the plurality of processing units are capable of being respectively installed; the plurality of connection circuits (26) connected respectively to the plurality of processing units; the plurality of circuit control devices (66) which the plurality of connection circuits are capable of being respectively attached to and detached from and which are capable of supplying the cells and the culture medium from the plurality of connection circuits to the plurality of processing units and moving a collection of cultured cells from the plurality of processing units to the plurality of connection circuits; and the sensor device (70) which is used in common with respect to the plurality of processing units, in order to measure components of the culture medium that is guided into the plurality of processing units, where each of the plurality of processing units includes the plurality of bioreactors (30).

In the above-described cell culturing system, the sensor device may be connected to the plurality of processing units or to the plurality of connection circuits, in a set state with the plurality of processing units installed respectively in the plurality of reactor installation devices.

In the above-described cell culturing system, there may further be provided the tank device (64) having the culture medium accommodation unit (74) configured to accommodate the culture medium, where the culture medium accommodation unit may be used in common with respect to the plurality of processing units, in order to supply the culture medium from the culture medium accommodation unit to the plurality of processing units via the plurality of connection circuits.

In the above-described cell culturing system, the culture medium accommodation unit may be configured to accommodate an amount of the culture medium necessary for culturing cells in the plurality of processing units.

In the above-described cell culturing system, the tank device may include the waste liquid accommodation unit (78) configured to accommodate the waste liquid, and the waste liquid accommodation unit may be used in common with respect to the plurality of processing units, in order to discharge the waste liquid from the plurality of processing units into the waste liquid accommodation unit via the plurality of connection circuits.

In the above-described cell culturing system, the tank device may include the cleaning solution accommodation unit (76) configured to accommodate the cleaning solution, and the cleaning solution accommodation unit may be used in common with respect to the plurality of processing units, in order to supply the cleaning solution from the cleaning solution accommodation unit to the plurality of processing units via the plurality of connection circuits.

In the above-described cell culturing system, the cleaning solution accommodation unit may be configured to accommodate an amount of the cleaning solution necessary for cleaning the plurality of processing units.

In the above-described cell culturing system, the sensor device may include the sensor unit (148) that measures the components of the culture medium, and the plurality of pumps (146) that guide the culture medium, which has flowed through the plurality of processing units, to the sensor unit.

In the above-described cell culturing system, there may further be provided the controller (16) that controls operation of the plurality of circuit control devices, where the controller may feedback control operation of the plurality of circuit control devices based on a measurement result of the sensor device.

In the above-described cell culturing system, each of the plurality of bioreactors may contain a plurality of the hollow fibers (32).

1. A cell culturing system comprising:
a plurality of processing units configured to perform culturing of cells, each of the plurality of processing units including a plurality of bioreactors;
a plurality of connection circuits configured to be electrically connected to the plurality of processing units, respectively;
a plurality of circuit control devices configured to be attachable to and detachable from the plurality of connection circuits, respectively, each of the circuit control devices of the plurality of circuit control devices configured to supply the cells and a culture medium from the plurality of connection circuits to the plurality of processing units and to move a collection of cultured cells from the plurality of processing units to the plurality of connection circuits; and
a sensor device configured to be used for each processing unit of the plurality of processing units and configured to measure components of the culture medium guided into each of the processing units of the plurality of processing units.

2. The cell culturing system of claim 1, wherein the cell culturing system further includes:
a plurality of reactor installation devices configured to receive the plurality of processing units.

3. The cell culturing system of claim 3, wherein the sensor device is configured to be electrically connected to the plurality of processing units or to the plurality of connection circuits, where the plurality of processing units are installed in the plurality of reactor installation devices, respectively.

4. The cell culturing system of claim 1, wherein the cell culturing system further includes:
a tank device including a culture medium accommodation unit configured to accommodate the culture medium.

5. The cell culturing system of claim 4, wherein the culture medium accommodation unit is configured to be used in common for each of the processing units of the plurality of processing units in order to supply the culture medium from the culture medium accommodation unit to the plurality of processing units via the plurality of connection circuits.

6. The cell culturing system of claim 4, wherein the culture medium accommodation unit is configured to accommodate an amount of the culture medium necessary for culturing cells in each of the processing units of the plurality of processing units.

7. The cell culturing system of claim 4, wherein the tank device further includes a waste liquid accommodation unit configured to accommodate a waste liquid.

8. The cell culturing system of claim 7, wherein the waste liquid accommodation unit is used for each of the processing units of the plurality of processing units in order to discharge the waste liquid from the plurality of processing units into the waste liquid accommodation unit via the plurality of connection circuits.

9. The cell culturing system of claim 4, wherein the tank device further includes a cleaning solution accommodation unit configured to accommodate a cleaning solution.

10. The cell culturing system of claim 9, wherein the cleaning solution accommodation unit is used for each of the processing units of the plurality of processing units in order to supply the cleaning solution from the cleaning solution accommodation unit to the plurality of processing units via the plurality of connection circuits.

11. The cell culturing system of claim 9, wherein the cleaning solution accommodation unit is configured to accommodate an amount of the cleaning solution necessary for cleaning each of the processing units of the plurality of processing units.

12. The cell culturing system of claim 1, wherein the sensor device includes:
a sensor unit configured to measure the components of the culture medium; and
a plurality of pumps configured to guide the culture medium from the plurality of processing units to the sensor unit.

13. The cell culturing system of claim 1, wherein the cell culturing system further includes:
a controller configured to control operation of the plurality of circuit control devices.

14. The cell culturing system of claim 13, wherein the controller is configured to control operation of the plurality of circuit control devices based on measurement results of the sensor device.

15. The cell culturing system of claim 1, wherein each of the plurality of bioreactors includes a plurality of hollow fibers.

16. A cell culturing system comprising:
a plurality of processing units configured to receive a culture medium and to culture cells, each of the of the plurality of processing units including a plurality of bioreactors; and
a sensor device configured to be used for each processing unit of the plurality of processing units and configured to measure components of the culture medium received in each processing unit of the plurality of processing units.

17. The cell culturing system of claim 16, wherein the cell culturing system further includes:
a plurality of reactor installation devices configured to receive the plurality of processing units, the plurality of processing units are installed in the plurality of reactor installation devices, respectively;
a plurality of connection circuits configured to be electrically connected to the plurality of processing units, respectively;
a plurality of circuit control devices configured to be attachable to and detachable from the plurality of connection circuits, respectively, each of the circuit control devices of the plurality of circuit control devices configured to supply the cells to be cultured and the culture medium from the plurality of connection circuits to the plurality of processing units and to move a collection of cultured cells from the plurality of processing units to the plurality of connection circuits.

18. The cell culturing system of claim 16, wherein the cell culturing system further includes:
a tank device having a culture medium accommodation unit configured to accommodate the culture medium, the culture medium accommodation unit being configured to be used in common for each of the processing units of the plurality of processing units in order to supply the culture medium from the culture medium accommodation unit to the plurality of processing units.

19. The cell culturing system of claim 16, wherein the tank device further includes a waste liquid accommodation unit configured to accommodate a waste liquid, the waste liquid accommodation unit being used for each of the processing units of the plurality of processing units in order to discharge the waste liquid from the plurality of processing units into the waste liquid accommodation unit.

20. The cell culturing system of claim 16, wherein the tank device further includes a cleaning solution accommodation unit configured to accommodate a cleaning solution, the cleaning solution accommodation unit being used for each of the processing units of the plurality of processing units in order to supply the cleaning solution from the cleaning solution accommodation unit to the plurality of processing units.
